(19) United States
(12) Patent Application Publication
(10) Pub. No.: US 2002/0122806 A1
(43) Pub. Date: Sep. 5, 2002

(54) Compositions and methods for in situ and in vivo imaging of cells and tissues

(76) Inventors: Arul M. Chinnaiyan, Canton, MI (US); Alnawaz Rehemtulla, Plymouth, MI (US); Brian D. Ross, Ann Arbor, MI (US)

Correspondence Address: GREGORY P. EINHORN, FISH & RICHARDSON P.C., Suite 500, 4350 La Jolla Village Drive, San Diego, CA, 92122, US

(21) Appl. No.: 09/734,628

(22) Filed: Mar. 5, 2001

(51) Int. Cl. A61K 39/395; C07H 21/04

(52) U.S. Cl. 424/178.1; 435/325; 536/23.5

(57) ABSTRACT

The invention provides compositions and methods for imaging cells and tissues in vivo and in situ. Compositions and methods are provided for administration to enhance the imaging of cells and tissues by, e.g., computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Thus, the invention provides compositions and methods for identifying sites of primary and metastatic tumors and tumor neovasculature.

### TECHNICAL FIELD

This invention generally pertains to the fields of medicine and non-invasive imaging. The invention provides compositions and methods for imaging cells, tissues and organs in vivo and in situ. In particular, compositions and methods are provided to enhance the imaging of cells and tissues by, e.g., computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Thus, the invention provides compositions and methods for imaging normal and abnormal tissues, including, e.g., sites of primary and metastatic tumors and tumor neovasculature.

### BACKGROUND

Neovascularization is essential for tumor growth and metastasis. It is now widely held that acquisition of an angiogenic phenotype is an early event in tumorigenesis, allowing tumors to grow beyond the size otherwise limited by the diffusion of oxygen and other nutrients through tissue. The identification of families of endogenous pro- and anti-angiogenic factors has fueled great excitement about therapeutic approaches aimed at interrupting tumor blood supply. Furthermore, proliferating endothelial cells forming tumor neovasculature express a number of proteins that are not found in mature vessels. These include endothelial cell surface alpha ν integrins, which tightly bind peptides containing the Arg-Gly-Asp (RGD) motif. Studies have found that tumor neovasculature endothelium can express tumor-specific molecules (see, e.g., Pasqualini (1997) Nature Biotechnol. 15:542-546). Phage displaying RGD peptides on their surface can selectively accumulate in tumor blood vessels and RGD peptides can inhibit metastasis in vivo (see, e.g., Fujii (1995) Biol. Pharm. Bull. 18:1681-1688; Saiki (1990) Jpn. J. Cancer Res. 81:1003-1011).

### SUMMARY

The invention provides compositions and methods for imaging cells and tissues in vivo and in situ; which can be particularly useful for imaging normal and abnormal tissue and organs, including sites of primary and metastatic tumors and tumor neovasculature.

The invention provides a chimeric molecule comprising a first domain comprising a fluorescent, bioluminescent or chemiluminescent polypeptide, or a heterologous (i.e., non-endogenous) kinase, and a second domain comprising a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide. In alternative aspects, the fluorescent or chemiluminescent polypeptide is luciferase, aequorin, obelin, mnemiopsin or berovin or equivalents or combinations thereof.

The heterologous (non-endogenous) kinase can be a non-mammalian kinase, e.g., a herpes simplex virus-1 thymidine kinase (HSV-1 TK).

In one aspect of the invention, the RGD motif-comprising polypeptide (and thus the chimeric molecule) specifically binds to a cell-specific polypeptide, a tissue-specific polypeptide or an organ-specific polypeptide. The cell-specific polypeptide can be a tumor-specific polypeptide. The cell-specific polypeptide can be expressed on tumor neovasculature, including vascular endothelium. The RGD motif-comprising polypeptide can comprise an integrin polypeptide or fragment thereof. The RGD motif-comprising polypeptide can comprise an CDCRGDCFC (SEQ ID NO:1) amino acid sequence.

In another aspect of the invention, the selectin can be an E-selectin. The selectin binding polypeptide can comprise an IELLQAR (SEQ ID NO:2) amino acid sequence.

In one aspect of the invention, the matrix metalloproteinase (MMP) is a gelatinase A or a gelatinase B. The matrix metalloproteinase binding polypeptide can comprise an CTTHWGFTLC (SEQ ID NO:3) amino acid sequence.

In one aspect of the invention the chondroitin sulfate proteoglycan comprises a high molecular weight (MW) human melanoma-associated antigen. The chondroitin sulfate proteoglycan binding polypeptide can comprise a TAASGVRSMH (SEQ ID NO:4) or LTLRWVGLMS (SEQ ID NO:5) sequence.

In one aspect of the invention, the chimeric molecule is capable of specifically binding to a lumen-expressed vascular endothelial cell composition, such as a protein, carbohydrate, proteoglycan; the composition may be expressed exclusively during tumor neovascularization.

The invention provides a pharmaceutical formulation comprising a chimeric molecule and a pharmaceutically acceptable excipient, wherein the chimeric molecule comprises a first domain comprising a fluorescent, bioluminescent or chemiluminescent polypeptide, or a kinase (e.g., a heterologous kinase), and a second domain comprising a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide, wherein the formulation is suitable for administration as an imaging enhancing agent (e.g., for intravenous injection) and the chimeric molecule is present is an amount sufficient to enhance a computer assisted tomography (CAT) image, a magnetic resonance spectroscopy (MRS) image, a magnetic resonance imaging (MRI) image, a positron emission tomography (PET) image, single-photon emission computed tomography (SPECT) image, or bioluminescence imaging (BLI) (when administered to an individual, or applied to a tissue or organ in situ, in a sufficient amount).

The invention provides a pharmaceutical formulation comprising a composition comprising a first domain comprising an imaging enhancing agent and a second domain comprising a polypeptide that binds to a cell, a tissue or an organ in a cell-, tissue-, or organ-specific manner, and, a pharmaceutically acceptable excipient suitable for administration as an imaging enhancing agent, wherein composition is not an antibody and is present is an amount sufficient to enhance a computer assisted tomography (CAT) image, a magnetic resonance spectroscopy (MRS) image, a magnetic resonance imaging (MRI) image, a positron emission tomography (PET) image, or a single-photon emission computed tomography (SPECT) image, bioluminescence imaging (BLI) or equivalent, when the pharmaceutical formulation is administered to an individual, or applied to a tissue or organ in situ, in a sufficient amount.

In one aspect of the pharmaceutical formulation, the image enhancing agent comprises a kinase, e.g., a heterologous kinase. The kinase can comprise a herpes simplex virus-1 thymidine kinase (HSV-1 TK).

In one aspect of the pharmaceutical formulation, the imaging enhancing agent comprises a bioluminescent or chemiluminescent polypeptide and the composition is a chimeric recombinant protein. The bioluminescent or chemiluminescent compound can comprise a luciferase, an aequorin, an obelin, a mnemiopsin, a berovin, a phenanthridinium ester, or an equivalent thereof or a combination thereof.

The imaging-enhancing agent of the pharmaceutical formulation can comprise a radioactive isotope, such as ¹³¹I, ¹²⁵I, ¹²³I, ¹⁸F, ¹¹C, ⁷⁵Br, ⁷⁶Br, ¹⁹F, ¹³C, ¹⁴C or ³H. The imaging-enhancing agent can comprise a paramagnetic compound, such as a polypeptide chelated to a metal, e.g., a metalloporphyrin. The paramagnetic compound can also comprise a monocrystalline nanoparticle, e.g., a nanoparticle comprising a lanthanide (e.g., Gd) or iron oxide; or, a metal ion comprising a lanthanide. Lanthanides include elements of atomic numbers 58 to 70, a transition metal of atomic numbers 21 to 29, 42 or 44, a Gd(III), a Mn(II), or an element comprising an Fe element. The paramagnetic compound can comprise a neodymium iron oxide (NdFeO₃) or a dysprosium iron oxide (DyFeO₃). The imaging-enhancing agent can comprise a synthetic compound, a peptidomimetic or a peptide (as used herein, the term polypeptide includes peptidomimetic and a peptide, as discussed below).

In another aspect of the pharmaceutical formulation, the compound of the second domain can comprise a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide.

The pharmaceutical formulation of the invention can further comprise a cytotoxic agent, such as an antitumor agent, an anti-angiogenic agent or a therapeutic radionuclide. The pharmaceutical composition can further comprise a lipid, such as a liposome. The pharmaceutically acceptable excipient of the pharmaceutical formulation can be a buffered saline, or equivalent. In one aspect, the concentration of the composition in the pharmaceutical formulation is about 1 to about 1000 μg/ml, or, about 10 to about 100 μg/ml.

The pharmaceutical formulation of the invention can further comprise a substrate for the bioluminescent or chemiluminescent polypeptide (e.g., an enzyme, such as luciferase) or the heterologous kinase (in one aspect the substrate is altered by the enzyme or kinase; it is the altered molecule that is detected). For example, the chemiluminescent polypeptide can be luciferase and the substrate is luciferin.

The invention also provides a nucleic acid encoding a chimeric polypeptide of the invention. In one aspect, the nucleic acid comprises an open reading frame operably linked to a promoter, wherein the open reading frame encodes a chimeric polypeptide of the invention. The invention also provides an expression vector comprising a nucleic acid encoding a chimeric polypeptide of the invention. The invention provides a cell, e.g., a transformed or a viral vector-infected cell, comprising a nucleic acid encoding a chimeric polypeptide of the invention. The cell can be a bacterial, a yeast, an insect, or a mammalian cell. The invention provides a recombinant chimeric polypeptide produced by a cell of the invention. The recombinant polypeptide can be isolated to varying degrees.

The invention also provides a method for in situ or in vivo imaging of a cell, a tissue, an organ or a full body comprising administration of a pharmaceutical formulation of the invention in an amount sufficient to enhance the image, wherein the image is generated by a computer assisted tomography (CAT) image, a magnetic resonance spectroscopy (MRS) image, a magnetic resonance imaging (MRI) image, a positron emission tomography (PET) image, a single-photon emission computed tomography (SPECT) image, a bioluminescence image (BLI) or equivalent.

The invention also provides a method for in situ or in vivo imaging of a cell, a tissue, an organ or a full body comprising the following steps: (a) providing a pharmaceutical formulation of the invention (b) providing an imaging device, wherein the imaging device is computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent; (c) administering the pharmaceutical formulation in an amount sufficient to generate the cell, tissue or body image; and, (d) imaging the distribution of the pharmaceutical formulation of step (a) with the imaging device, thereby imaging the cell, tissue or body.

In one aspect of the method of the invention, the pharmaceutical formulation is administered to a human, such as a cancer patient or a patient suspected of having or being screened for cancer. The tissue can be a tumor tissue, such as a solid tumor, a metastasis or the neovasculature of a tumor. The pharmaceutical formulation can be administered intravenously. The image can be taken between about 2 minutes and about 24 hours after administering the pharmaceutical formulation.

The methods of the invention can fturther comprise providing a reagent (a substrate) for the bioluminescent or chemiluminescent polypeptide or the heterologous kinase, and administering the reagent with or after administration of the bioluminescent or chemiluminescent polypeptide or the heterologous kinase. The chemiluminescent polypeptide can be luciferase and the reagent luciferin.

The invention provides a method for in vivo imaging tumor neovasculature in an individual comprising the following steps: (a) providing a pharmaceutical formulation of the invention; (b) providing an imaging device, wherein the imaging device is computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent; (c) administering the pharmaceutical formulation in an amount sufficient to image the tumor neovasculature; and, (d) imaging the distribution of the pharmaceutical formulation of step (a) with the imaging device, thereby imaging the tumor neovasculature.

The invention provides a method for in situ or in vivo screening for an anti-tumor agent by imaging a tumor neovasculature in an individual comprising the following steps: (a) providing a composition comprising a chimeric polypeptide as set forth in claim 1 or a pharmaceutical formulation as set forth in claim 18, and a test compound; (b) providing an imaging device, wherein the imaging device is computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent; (c) administering the composition of step (a) in an amount sufficient to image the tumor neovasculature and imaging the distribution of the composition with the imaging device, thereby imaging the tumor neovasculature; (d) administering the test compound; and, (e) imaging the distribution of the composition with the imaging device, thereby imaging the tumor neovasculature, wherein a decrease in the amount of tumor neovasculature indicates that the compound is an anti-tumor or an anti-angiogenic agent.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 shows the chimeric RGD-luciferase protein attached to the surface of cultured cells as detected using BLI, as described in detail in Example 1, below.

FIG. 2 shows a nude mouse with an orthotopic mammary tumor imaged with an in vivo bioluminescent imaging system after injection of RDG-luciferase, as described in detail in Example 1, below. The presence of the tumor was detected by the emission of luciferase-produced photons from the tumor site (see arrow).

### DETAILED DESCRIPTION

The invention provides compositions and methods for imaging cells, tissues and organs in vivo and in situ. The compositions and methods of the invention are used to identify sites of primary and metastatic tumors and tumor neovasculature. Thus, the invention also provides a non-invasive means to evaluate the effectiveness of a therapy for a disorder of cell growth, such as cancer. The compositions and methods enhance the imaging of cells, tissues, organs, entire bodies and angiogenesis by, e.g., computer assisted tomography (CAT), magnetic resonance spectroscopy (MRS), magnetic resonance imaging (MRI), positron emission tomography (PET), single-photon emission computed tomography (SPECT), or bioluminescence imaging (BLI).

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

The term “antibody” or “Ab” includes both intact antibodies having at least two heavy (H) chains and two light (L) chains inter-connected by disulfide bonds and antigen binding fragments thereof, or equivalents thereof, either isolated from natural sources, recombinantly generated or partially or entirely synthetic. Examples of antigen binding fragments include, e.g., Fab fragments, F(ab′)2 fragments, Fd fragments, dAb fragments, isolated complementarity determining regions (CDR), single chain antibodies, chimeric antibodies, humanized antibodies, human antibodies made in non-human animals (e.g., transgenic mice) or any form of antigen binding fragment.

The term “non-endogenous kinase” or “heterologous kinase” means a kinase not normally associated with a cell or tissue, as described in further detail, below. In some aspects, an exemplary kinase used in the compositions and methods of the invention includes herpes simplex virus-1 thymidine kinase (HSV-1 TK). For example, in the methods of the invention a chimeric polypeptide comprising a domain comprising a non-mammalian kinase is administered to a mammal, e.g., a human.

As used herein, the term “bioluminescence imaging” or “BLI” includes all bioluminescence, fluorescence or chemiluminescence or other photon detection systems and devices capable of detecting bioluminescence, fluorescence or chemiluminescence or other photon detection systems. Since light can be transmitted through mammalian tissues at a low level, bioluminescent and fluorescent proteins can be detected externally using sensitive photon detection systems; see, e.g., Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. An exemplary photodetector device is an intensified charge-coupled device (ICCD) camera coupled to an image processor. See, e.g., U.S. Pat. No. 5,650,135. Photon detection devices are manufactured by, e.g., Xenogen, Hamamatsue.

As used herein, a “computer assisted tomography (CAT)” or a “computerized axial tomography (CAT)” incorporates all computer-assisted tomography imaging systems or equivalents and devices capable of computer assisted tomography imaging. The methods of the invention can be practiced using any such device, or variation of a CAT device or equivalent, or in conjunction with any known CAT methodology. See, e.g., U.S. Pat. Nos. 6,151,377; 5,946,371; 5,446,799; 5,406,479; 5,208,581; 5,109,397. Animal imaging modalities are also included, such as MicroCAT™ (ImTek, Inc.).

As used herein, “positron emission tomography imaging (PET)” incorporates all positron emission tomography imaging systems or equivalents and all devices capable of positron emission tomography imaging. The methods of the invention can be practiced using any such device, or variation of a PET device or equivalent, or in conjunction with any known PET methodology. See, e.g., U.S. Pat. Nos. 6,151,377; 6,072,177; 5,900,636; 5,608,221; 5,532,489; 5,272,343; 5,103,098. Animal imaging modalities are included, e.g. micro-PETs (Corcorde Microsystems, Inc.).

As used herein, “single-photon emission computed tomography (SPECT) device” incorporates all single-photon emission computed tomography imaging systems or equivalents and all devices capable of single-photon emission computed tomography imaging. The methods of the invention can be practiced using any such device, or variation of a SPECT device or equivalent, or in conjunction with any known SPECT methodology. See, e.g., U.S. Pat. Nos. 6,115,446; 6,072,177; 5,608,221; 5,600,145; 5,210,421; 5,103,098. Animal imaging modalities are also included, such as micro-SPECTs.

As used herein, “magnetic resonance imaging (MRI) device” incorporates all magnetic resonance imaging systems or equivalents and all devices capable of magnetic resonance imaging. The methods of the invention can be practiced using any such device, or variation of an MRI device or equivalent, or in conjunction with any known MRI methodology. In magnetic resonance methods and apparatus a static magnetic field is applied to a tissue or a body under investigation in order to define an equilibrium axis of magnetic alignment in a region of interest. A radio frequency field is then applied to that region in a direction orthogonal to the static magnetic field direction in order to excite magnetic resonance in the region. The resulting radio frequency signals are detected and processed. The exciting radio frequency field is applied. The resulting signals are detected by radio-frequency coils placed adjacent the tissue or area of the body of interest. See, e.g., U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279. MRI and supporting devices are manufactured by, e.g., Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. Animal imaging modalities are also included, such as micro-MRIs.

As used herein, the terms “computer” and “processor” are used in their broadest general contexts and incorporate all such devices. The methods of the invention can be practiced using any computer/processor and in conjunction with any known software or methodology. For example, a computer/processor can be a conventional general-purpose digital computer, e.g., a personal “workstation” computer, including conventional elements such as microprocessor and data transfer bus. The computer/processor can further include any form of memory elements, such as dynamic random access memory, flash memory or the like, or mass storage such as magnetic disc optional storage.

As used herein, “chondroitin sulfate proteoglycan” includes those proteoglycan molecules expressed by immature cells and tumor cells, including the human melanoma proteoglycan also known as the high molecular weight melanoma-associated antigen, see, e.g., Desai (1998) Cancer Res. 58:2417-2425; Chattopadhyay (1991) Cancer Res. 51:3183-3192. Chondroitin sulfate proteoglycan binding polypeptides are discussed below.

As used herein, “fluorescent,” “bioluminescent” and “chemiluminescent” polypeptides include all known polypeptides known to be fluorescent, bioluminescent or chemiluminescent, or, acting as enzymes on a specific substrate (reagent), can generate a fluorescent, bioluminescent or chemiluminescent molecule. They include, e.g., isolated and recombinant luciferases, aequorin, obelin, mnemiopsin, berovin and variations thereof and combinations thereof, as discussed in detail, below. The pharmaceutical formulation of the invention can further comprise a substrate for the bioluminescent or chemiluminescent polypeptide. For example, the chemiluminescent polypeptide can be luciferase and the reagent luciferin. The substrate can be administered before, at the same time (e.g., in the same formulation), or after administration of the chimeric polypeptide (including the enzyme).

As used herein, “lanthanide metal ions” include, e.g., lanthanum [La], cerium [Ce], praseodyme [Pr], gadolinium [Gd], dysprosium [Dy], ytterbium [Yb], and lutetium [Lu]. Non-lanthanides that can be imaged are also used in the methods of the invention and include lead [Pb] and bismuth [Bi]. See, e.g. Krause (1996) Invest. Radiol. 31:502-511.

As used herein, “matrix metalloproteinase (MMP)” includes interstitial collagenases, stromelysins, gelatinases and membrane-type metalloproteinases and MMPs secreted by cancer cells. These later MMPs degrade extracellular matrices and allow for tumor infiltration of tissues and metastasis; see, e.g., Yip (1999) Invest. New Drugs 17:387-399. MMPs and MMP-binding polypeptides are discussed in detail, below.

As used herein, “selectin” includes L-, E-, and P-selectins, which are membrane-anchored, C-type lectins that initiate tethering and rolling of flowing leukocytes on endothelial cells, platelets, or other leukocytes during inflammation. Selectins bind to sialylated, fucosylated, sulfated glycans on glycoproteins, glycolipids, or proteoglycans. E-selectin, which are expressed on vascular endothelial cells, can bind to carbohydrate determinants, e.g., sialyl Lewis A and sialyl Lewis X, which are frequently expressed on human cancer cells. See, e.g., McEver (1997) Glycoconj. J. 14:585-591; Kannagi (1997) Glycoconj. J. 14:577-584. Selectin-binding polypeptides are discussed in detail, below.

The term “pharmaceutical composition” refers to a composition suitable for pharmaceutical use in a subject (including human or veterinary). The pharmaceutical compositions of this invention are formulations that comprise a pharmacologically effective amount of a composition comprising, e.g., a chimeric composition, a recombinant polypeptide, a nucleic acid encoding a chimeric polypeptide of the invention, a vector comprising a nucleic acid of the invention, or a cell of the invention, and a pharmaceutically acceptable carrier. The pharmaceutical formulation of the invention can further comprise a substrate for the bioluminescent or chemiluminescent polypeptide. For example, the chemiluminescent polypeptide can be luciferase and the reagent luciferin. Alternatively, the substrate reagent can be co-administered or administered before or after the chimeric polypeptide (enzyme) formulation.

As used herein, the term “promoter” includes all sequences capable of driving transcription of a coding sequence in an expression system. Thus, promoters used in the constructs of the invention include cis-acting transcriptional control elements and regulatory sequences that are involved in regulating or modulating the timing and/or rate of transcription of a nucleic acid of the invention. As used herein, the term “operably linked,” refers to a functional relationship between two or more nucleic acid (e.g., DNA) segments. Typically, it refers to the functional relationship of a transcriptional regulatory sequence to a transcribed sequence. As used herein, “recombinant” refers to a polynucleotide synthesized or otherwise manipulated in vitro (e.g., “recombinant polynucleotide” ), to methods of using recombinant polynucleotides to produce gene products in cells or other biological systems, or to a polypeptide (“recombinant protein” ) encoded by a recombinant polynucleotide.

The term “nucleic acid” or “nucleic acid sequence” refers to a deoxy-ribonucleotide or ribonucleotide oligonucleotide, including single- or double-stranded, or coding or non-coding (e.g., “antisense”) forms. The term encompasses nucleic acids, i.e., oligonucleotides, containing known analogues of natural nucleotides. The term also encompasses nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156.

As used herein the terms “polypeptide,” “protein,” and “peptide” are used interchangeably and include compositions of the invention that also include “analogs,” or “conservative variants” and “mimetics” (e.g., “peptidomimetics”) with structures and activity that substantially correspond to the polypeptides of the invention, including the peptide sequences as set forth in SEQ ID NO:1, SEQ ID NO:2, SEQ ID NO:3 and SEQ ID NO:4. Thus, the terms “conservative variant” or “analog” or “mimetic” also refer to a polypeptide or peptide which has a modified amino acid sequence, such that the change(s) do not substantially alter the polypeptide's (the conservative variant's) structure and/or activity (e.g., binding specificity), as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity. Conservative substitution tables providing functionally similar amino acids are well known in the art. For example, one exemplary guideline to select conservative substitutions includes (original residue followed by exemplary substitution): ala/gly or ser; arg/lys; asn/gIn or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W); (see also, e.g., Creighton (1984) Proteins, W. H. Freeman and Company; Schulz and Schimer (1979) Principles of Protein Structure, Springer-Verlag). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides of the invention (e.g., ability to bind to integrins, selecting, MMPs, chondroitin sulfate proteoglycans, etc.). The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetics' structure and/or activity. As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups. other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for—C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY). A polypeptide can also be characterized as a mimetic by containing all or some non-natural residues in place of naturally occurring amino acid residues; non-natural residues are well described in the scientific and patent literature.

### FLUORESCENT, BIOLUMINESCENT OR CHEMILUMINESCENT POLYPEPTIDES

The invention provides a chimeric molecule, e.g., a recombinant polypeptide, and a pharmaceutical composition, comprising a fluorescent, bioluminescent or chemiluminescent polypeptide. As defined above, these polypeptides include enzymes that act on a specific reagent to generate a molecule that can be imaged (e.g., luciferase reacting with luciferin in situ). In alternative aspects, these polypeptides include, e.g., luciferase, aequorin, halistaurin, phialidin, obelin, mnemiopsin or berovin, or, equivalent photoproteins, and combinations thereof. The compositions and methods of the invention also include recombinant forms of these polypeptides as recombinant chimeric or “fusion” proteins, including chimeric nucleic acids and constructs encoding them. Methods of making recombinant forms of these polypeptides are well known in the art, e.g., luciferase reporter plasmids are described, e.g., by Everett (1999) J. Steroid Biochem. Mol. Biol. 70:197-201. Sala-Newby (1998) Immunology 93:601-609, described used of a recombinant cytosolic fusion protein of firefly luciferase and aequorin (luciferase-aequorin). The Ca²⁺-activated photoprotein obelin is described by, e.g., Dormer (1978) Biochim. Biophys. Acta 538:87-105; and, recombinant obelin is described by, e.g., Illarionov (2000) Methods Enzymol. 305:223-249. The photoprotein mnemiopsin is described by, e.g., Anctil (1984) Biochem J. 221:269-272. The monomeric Ca2+-binding protein aequorin is described by, e.g., Kurose (1989) Proc. Natl. Acad. Sci. USA 86:80-84; Shimomura (1995) Biochem. Biophys. Res. Commun. 211:359-363. The aequorin-type photoproteins halistaurin and phialidin are described by, e.g., Shimomura (1985) Biochem J. 228:745-749. Ward (1975) Proc. Natl. Acad. Sci USA 72:2530-2534, describes the purification of mnemiopsin, aequorin and berovin. The recombinant fluorescent, bioluminescent or chemiluminescent chimeric polypeptides of the invention can be made by any method, see, e.g., U.S. Pat. No. 6,087,476, that describes making recombinant, chimeric luminescent proteins. U.S. Pat. Nos. 6,143,50; 6,074,859; 6,074,859, 5,229,285, describe making recombinant luminescent proteins. The fluorescent or chemiluminescent activity of the chimeric recombinant polypeptides of the invention can be assayed, e.g., using assays described in, e.g., U.S. Pat. Nos. 6,132,983; 6,087,476; 6,060,261; 5,866,348; 5,094,939; 5,744,320. Various photoproteins that can be used in compositions of the invention are described in, e.g., U.S. Pat. Nos. 5,648,218; 5,360,728; 5,098,828.

### RGD MOTIF-COMPRISING POLYPEPTIDES

The invention provides a chimeric molecule, e.g., a recombinant polypeptide, and a pharmaceutical composition, comprising a fluorescent or chemiluminescent polypeptide and an RGD motif-comprising polypeptide, such as the peptide comprising an CDCRGDCFC (SEQ ID NO:1) amino acid sequence. The tripeptidic sequence Arg-Gly-Asp, or “RGD,” is often the primary site of recognition by integrins that are expressed on tumor cells and are responsible for tumor invasion and metastasis. Tumor cell-expressing polypeptides that recognize RGD-comprising polypeptides mediate tumor cell adhesion to the components of extracellular matrix and basement membrane (e.g. laminin, vitronectin and fibronectin). Thus, RGD-comprising polypeptides mediate the invasion and migration of tumor cells. See, e.g., Fujii (1995) Biol. Pharm. Bull. 18:1681-1688; Saiki (1990) Jpn. J. Cancer Res. 81:1003-1011. Pasqualini (1997) Nat. Biotechnol. 15:542-546 showed that alpha v integrins present in tumor blood vessels can bind circulating RGD-expressing peptide ligands selective for these integrins. Thus, imaging of cells that can specifically bind to RGD-expressing peptide and polypeptide ligands in vivo can identify tumor cells and tumor blood vessels.

### SELECTIN BINDING COMPOSITIONS

The invention provides a chimeric molecule, e.g., a recombinant polypeptide, and a pharmaceutical composition, comprising a fluorescent or chemiluminescent polypeptide and a selectin- (e.g., E-selectin-) binding polypeptide, such as the peptide comprising an IELLQAR (SEQ ID NO:2) amino acid sequence. In nature, selectin-binding ligands include carbohydrates, such as sialyl Lewis X, that bind in a calcium-dependent manner. Significantly, some selectin carbohydrate ligands are expressed on tumor cells. Aberrant expression of cell surface carbohydrates such as sialyl Lewis X is associated with tumor formation and metastasis (see, e.g., Ohyama (1999) EMBO J. 18:1516-1525). Thus, imaging of ligands that can specifically bind to selectin in vivo can identify cells, e.g., immune cells, such as NK cells, that can target tumor cells.

During metastasis, tumor cells adhere to vascular endothelia. E-selectin is an adhesive protein expressed by cytokine-activated endothelium that can support adhesion of some cancer cells (e.g., colon cancer cells) through the recognition of specific carbohydrate ligands that can selectively bind to a selectin polypeptide can be done by routine screening using in vitro and in vivo techniques, including the use of high throughput screening of various peptide and other combinatorial libraries. For example, phage display libraries expressing libraries of peptides can be routinely screened for selectin-binding members. Alternatively, screening of selectin-binding compounds can also be done indirectly using “anti-idiotype” strategies; for example, antibodies that bind to carbohydrate ligands that are selectin ligands can be screened by, e.g., combinatorial or phage display libraries. See, e.g., Fukuda (2000) Cancer Res. 60:450-456, who used to method to identify the peptide IELLQAR (SEQ ID NO:2) as a selectin-binding ligand.

### MATRIX METALLOPROTEINASE BINDING COMPOSITIONS

The invention provides a chimeric molecule, e.g., a recombinant polypeptide, and a pharmaceutical composition, comprising a fluorescent or chemiluminescent polypeptide and a matrix metalloproteinase- (MMP-) binding polypeptide, such as the peptide comprising an CTTHWGFTLC (SEQ ID NO:3) amino acid sequence. Matrix metalloproteinases (MMPs) are secreted by cancer cells having the effect of degrading extracellular matrices and allow for tumor infiltration of tissues and metastasis (see, e.g., Minamitani (2000) Kurume Med. J. 47:115-124). Examples of tumor expressed MMPs are gelatinase A (also called matrix metalloproteinase 2, or MMP-2) and gelatinase B (also called matrix metalloproteinase 9 or MMP-9). Thus, imaging of cells that can specifically bind to MMP polypeptides in vivo can identify tumor cells and site of metastasis.

Identification of compounds that can selectively bind to MMP polypeptides can be done by routine screening using in vitro and in vivo techniques, including the use of high throughput screening of various peptide and other combinatorial libraries. For example, phage display libraries expressing libraries of peptides can be routinely screened for MMP-binding members. An exemplary technique is described by Koivunen (1999) Nat. Biotechnol. 17:768-774, who isolated specific of MMP-2 and MMP-9 gelatinase inhibitors from phage display peptide libraries, including the synthetic peptide CTTHWGFTLC (SEQ ID NO:3). Tamaki (1995) Chem. Pharm. Bull. (Tokyo) 43:1883-1893, also describes an assay to screen for inhibitors of gelatinase. See also U.S. Pat. Nos. 6,140,099; 6,114,568; 6,093,398; 5,595,885.

### PROTEOGLYCAN-BINDING PEPTIDES TARGET TUMOR NEOVASCULATURE

The invention provides a chimeric molecule, e.g., a recombinant polypeptide, and a pharmaceutical composition, comprising a fluorescent or chemiluminescent polypeptide and a chondroitin sulfate proteoglycan-binding polypeptide, such as a peptide comprising the sequence TAASGVRSMH (SEQ ID NO:4) or LTLRWVGLMS (SEQ ID NO:5). Chondroitin sulfate proteoglycans can be expressed by immature cells and tumor cells. For example, a human melanoma proteoglycan, also known as the high molecular weight melanoma-associated antigen, has been identified; see, e.g., Desai (1998) Cancer Res. 58:2417-2425; Chattopadhyay (1991) Cancer Res. 51:3183-3192. Another melanoma proteoglycan is NG2, a rat homologue of this human melanoma proteoglycan. NG2 is a 500 kDa integral membrane chondroitin sulfate proteoglycan. It is found on the surfaces of several different types of immature cells and is most prominent in immature blood vessels during development. NG2 is associated with multipotential glial precursor cells (O2A progenitor cells), chondroblasts of the developing cartilage, brain capillary endothelial cells, aortic smooth muscle cells, skeletal myoblasts and human melanoma cells (see, e.g., Levine (1996) Perspect. Dev. Neurobiol. 3:245-259). In adults, NG2 is expression can again be detected within newly forming blood vessels. It is not clear if expression of NG2 within the neovasculature can be attributed to endothelial cells or mesenchymal cells of the vasculature (smooth muscle cells or pericytes in microvessels). In adult animals, the expression of NG2 can be restricted to tumor cells and angiogenic tumor vasculature, making these proteoglycans targets for directing tumor imaging agents to relevant sites.

Identification of compounds that can selectively bind to tumor-expressing chondroitin sulfate proteoglycans can be done by routine screening using in vitro and in vivo techniques, including the use of high throughput screening of various peptide and other combinatorial libraries. For example, phage display libraries expressing libraries of peptides can be routinely screened for proteoglycan-binding members. An exemplary technique is described by Burg (1999) Cancer Res. 59:2869-2874, who identified specific NG2-binding peptides by screening a phage-displayed random peptide library on purified NG2, including the decapeptides TAASGVRSMH (SEQ ID NO:4) and LTLRWVGLMS (SEQ ID NO:5).

### NON-ENDOGENOUS KINASES

The invention provides a chimeric polypeptide having a first domain comprising a non-endogenous kinase, such as a herpes simplex virus-1 thymidine kinase (HSV-1 TK), and a second domain comprising an RGD motif-comprising polypeptide; a selectin-binding polypeptide; a matrix metalloproteinase (MMP)-binding polypeptide or a chondroitin sulfate proteoglycan-binding polypeptide. The chimeric polypeptide can be used as a reporter for administration in quantitative assays to non-invasively image expression of the polypeptide living animals using MRI, PET, SPECT, BRI and the like. For example, after administration of the chimeric polypeptide of the invention, a kinase substrate (a “reporter probe”) is administered, e.g., the positron-emitting 8-[18F] fluoroganciclovir (FGCV). In one aspect, the herpes simplex virus 1 thymidine kinase enzyme (HSV1-TK) is the kinase. Adenovirus-directed hepatic expression of the HSV-1 TK gene in living mice has been shown to be detectable by PET. See, e.g., Tjuvajev (1995) Cancer Res. 55:6126-6132; Gambhir (1999) Proc. Natl. Acad. Sci. USA 96:2333-2338; Gambhir (2000) Proc. Natl. Acad. Sci. USA 97:2785-2790; Gambhir (2000) Neoplasia 2:118-138; MacLaren (2000) Biol. Psychiatry 48:337-348; Schwimmer (2000) Q. J. Nucl. Med. 44:153-167; Yu (2000) Nat. Med. 6:933-937.

### IN VIVO BIOLUMINESCENT IMAGING

The invention provides compositions and methods to enhance the imaging of cells and tissues by, e.g., bioluminescence imaging (BLI). In vivo Bioluminescent Imaging (BLI) is a relatively new imaging modality; see discussion above and, e.g., Contag (2000) Neoplasia 2:41-52. This modality consists of the detection of a photoprotein (i.e., an optical reporter), such as luciferase from the firefly, using a sensitive photon detection system. The number of photons emitted from cells expressing the photoprotein (e.g., luciferase) can be quantitatively detected and overlayed (projected) onto a visual picture of the animal (including humans). This imaging approach provides a two-dimensional image data set and thus provides some spatial information as to the origin of the signal within the animal. An exciting aspect of BLI is its excellent sensitivity along with its ability to report on “molecular events” using specifically designed luciferase reporter constructs.

### CONTRAST AGENTS AND MAGNETIC IMAGING OF BRAIN TUMORS

The invention provides pharmaceutical formulations comprising the chimeric molecules of the invention that can further comprise imaging contrast agents (see, e.g., U.S. Pat. No. 4,731,239). The pharmaceutical formulations and/or the contrast agents can be administered by nanoencapsulation, e.g., by hydrogel nanoparticles (and liposomes, which are discussed below). Nanoencapsulation can be used to manipulate the environment surrounding the pharmaceutical formulation and/or the contrast agent. Although the contrast between healthy and abnormal tissues is strong, there exists considerable overlap of magnetic resonance imaging (MRI) T₁ and T₂ signals in all tissues. This physical property of biological tissues renders necessary the use of contrast agents for adequate resolution of many lesions: in particular, the diffuse margins of some lesions. Contrast agents for magnetic resonance imaging typically affect the protons on adjacent water molecules shortening either the T₁ or T₂ signals generated in the magnetic field. The most important factor in enhancement of relaxation is the difference between T₁ and T₂. There must be direct contact between protons and the magnetic parts of the contrast agent in order to shorten the T₁ component significantly. This effect can be clearly observed when gadolinium chelates are encapsulated in liposomes with resulting weakening of the T₁ signal. Weakening of the T₁ signal is thought to be due to the reduced access of water to the cavity of the liposome.

Enhancement of T₂ effects, however, requires clustering of the contrast agent and proximity to each other. This clustering of magnetic contrast agent exerts a greater influence over a much larger localized field. Thus incorporation into liposomes increases the proximity of T₂ contrast agents and enhances their effectiveness. Incorporation of contrast agents into the body of hydrogel nanoparticles has with it the potential advantages of both immobilizing and clustering the contrast agent and providing a material through which water can freely diffuse.

The pharmaceutical compositions of the invention can further comprise monocrystalline iron oxide nanoparticles (MION), which have been successfully used in a variety of biological and clinical applications. MION has an average diameter of approximately 18 to 24 nm and thus are able to penetrate endothelial fenestrations throughout the body and are cleared through the reticuloendothelial system and are disposed of by hepatic metabolism of iron. MION has excellent contrast characteristics in vivo and out-performs the most effective dendrimer-conjugated contrast agents. Although conjugation of MION to antibodies has been achieved, this strategy leaves considerable room for improvement with regard to contrast enhancement and specificity/uniformity of targeting. This invention provides improved strategies for efficient biotargeting of multifinctional imaging and therapeutic nanodevices.

### GOLD AND METAL NANOPARTICLES

The chimeric polypeptides of the invention can also be associated with gold or other equivalent metal particles (such as nanoparticles), either attached to the nanoparticles or as a further composition in the pharmaceutical formulations of the invention. Any metal particle system can be used, e.g., the gold nanoparticle Nanogold™ (Nanoprobes, Yaphank, N.Y.), which can be covalently attached to peptides via, e.g., a sulfo-succinimidyl-4-N-maleimido-cyclohexane-1-carboxylate (sulfo-SMCC) linkage (see, e.g., Du (1998) Neuroscience 84:37-48).

### POLYPEPTIDES AND PEPTIDES

The invention provides a chimeric polypeptide comprising a fluorescent, bioluminescent or chemiluminescent domain and an RGD motif-comprising polypeptide; a selectin-binding polypeptide; a matrix metalloproteinase (MMP)-binding polypeptide or a chondroitin sulfate proteoglycan-binding polypeptide. These chimeric polypeptides comprise sequences as set forth in SEQ ID NO:1, SEQ ID NO:2, SEQ ID NO:3 or SEQ ID NO:4 (as noted above, the term polypeptide includes peptides and peptidomimetics, etc.). Polypeptides and peptides of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art.

Polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer). The skilled artisan will recognize that individual synthetic residues and polypeptides incorporating mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Polypeptides incorporating mimetics can also be made using solid phase synthetic procedures, as described, e.g., by Di Marchi, et al., U.S. Pat. No. 5,422,426. Peptides and peptide mimetics of the invention can also be synthesized using combinatorial methodologies. Various techniques for generation of peptide and peptidomimetic libraries are well known, and include, e.g., multipin, tea bag, and split-couple-mix techniques; see, e.g., al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymol. 267:220-234. Modified peptides of the invention can be further produced by chemical modification methods, see, e.g., Belousov (1997) Nucleic Acids Res. 25:3440-3444; Frenkel (1995) Free Radic. Biol. Med. 19:373-380; Blommers (1994) Biochemistry 33:7886-7896.

Peptides and polypeptides of the invention can also be synthesized and expressed as chimeric or “fusion” proteins with one or more additional domains linked thereto for, e.g., to more readily isolate a recombinantly synthesized peptide, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between the purification domain and GCA-associated peptide or polypeptide can be useful to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-14). The histidine residues facilitate detection and purification while the enterokinase cleavage site provides a means for purifying the epitope from the remainder of the fusion protein.

### NUCLEIC ACIDS AND EXPRESSION VECTORS

This invention provides nucleic acids encoding the chimeric polypeptides of the invention. As the genes and vectors of the invention can be made and expressed in vitro or in vivo, the invention provides for a variety of means of making and expressing these genes and vectors. One of skill will recognize that desired phenotypes associated with altered gene activity can be obtained by modulating the expression or activity of the genes and nucleic acids (e.g., promoters) within the vectors of the invention. Any of the known methods described for increasing or decreasing expression or activity can be used for this invention. The invention can be practiced in conjunction with any method or protocol known in the art, which are well described in the scientific and patent literature.

The nucleic acid sequences of the invention and other nucleic acids used to practice this invention, whether RNA, cDNA, genomic DNA, vectors, viruses or hybrids thereof, may be isolated from a variety of sources, genetically engineered, amplified, and/or expressed recombinantly. Any recombinant expression system can be used, including, in addition to bacterial cells, e.g., mammalian, yeast, insect or plant cell expression systems.

Alternatively, these nucleic acids can be synthesized in vitro by well-known chemical synthesis techniques, as described in, e.g., Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., generating mutations in sequences, subcloning, labeling probes, sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

### FORMULATION AND ADMINISTRATION PHARMACEUTICALS

The invention provides pharmaceutical formulations comprising the chimeric molecules of the invention and a pharmaceutically acceptable excipient suitable for administration an imaging enhancing agents, and methods for making and using these compositions. These pharmaceuticals can be administered by any means in any appropriate formulation. Routine means to determine drug regimens and formulations to practice the methods of the invention are well described in the patent and scientific literature. For example, details on techniques for formulation, dosages, administration and the like are described in, e.g., the latest edition of Remington's Pharmaceutical Sciences, Maack Publishing Co, Easton Pa.

The formulations of the invention can include pharmaceutically acceptable carriers that can contain a physiologically acceptable compound that acts, e.g., to stabilize the composition or to increase or decrease the absorption of the agent and/or pharmaceutical composition. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of any co-administered agents, or excipients or other stabilizers and/or buffers. Detergents can also used to stabilize the composition or to increase or decrease the absorption of the pharmaceutical composition. Other physiologically acceptable compounds include wetting agents, emulsifying agents, dispersing agents or preservatives that are particularly useful for preventing the growth or action of microorganisms. Various preservatives are well known, e.g., ascorbic acid. One skilled in the art would appreciate that the choice of a pharmaceutically acceptable carrier, including a physiologically acceptable compound depends, e.g., on the route of administration and on the particular physio-chemical characteristics of any co-administered agent.

In one aspect, the composition for administration comprises a chimeric polypeptide of the invention in a pharmaceutically acceptable carrier, e.g., an aqueous carrier. A variety of carriers can be used, e.g., buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected.

The pharmaceutical formulations of the invention can be administered in a variety of unit dosage forms, depending upon the particular cell or tissue or cancer to be imaged, the general medical condition of each patient, the method of administration, and the like. Details on dosages are well described in the scientific and patent literature, see, e.g., the latest edition of Remington's Pharmaceutical Sciences. The exact amount and concentration of chimeric molecule or pharmaceutical of the invention and the amount of formulation in a given dose, or the “effective dose” can be routinely determined by, e.g., the clinician. The “dosing regimen,” will depend upon a variety of factors, e.g., whether the cell or tissue or tumor to be imaged is disseminated or local, the general state of the patient's health, age and the like. Using guidelines describing alternative dosaging regimens, e.g., from the use of other imaging contrast agents, the skilled artisan can determine by routine trials optimal effective concentrations of pharmaceutical compositions of the invention. For example, contrast-enhanced CT studies of the head and neck are performed frequently using contrast material volumes of about 30 g iodine and a scan delay of 30 to 45 seconds, see, e.g., Groell (1999) AJNR Am. J. Neuroradiol. 20:1732-1736. Desser (1999) Acad. Radiol. 6:176-183, described use of three different doses of liposomal iodixanol to image livers by CAT. Hamm (1994) J. Magn. Reson. Imaging 4:659-668, described use of various dosages of superparamagnetic iron oxide to MRI image livers and spleens. In one aspect, the concentration of the composition in the pharmaceutical formulation is about 1 to about 1000 μg/ml, or, 10 to about 100 μg/ml. However, higher or lower concentrations are used when appropriate and the invention is not limited by any particular dosage range.

The pharmaceutical compositions of the invention (e.g., chimeric polypeptides) can be delivered by any means known in the art systemically (e.g., intravenously), regionally, or locally (e.g., intra- or peri-tumoral or intracystic injection, e.g., to image bladder cancer) by, e.g., intraarterial, intratumoral, intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa), intra-tumoral (e.g., transdermal application or local injection). For example, intra-arterial injections can be used to have a “regional effect,” e.g., to focus on a specific organ (e.g., brain, liver, spleen, lungs). For example, intra-hepatic artery injection or intra-carotid artery injection. If it is desired to deliver the preparation to the brain, it can be injected into a carotid artery or an artery of the carotid system of arteries (e.g., occipital artery, auricular artery, temporal artery, cerebral artery, maxillary artery, etc.).

The pharmaceutical formulations of the invention can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Therapeutic compositions can also be administered in a lipid formulation, e.g., complexed with liposomes or in lipid/nucleic acid complexes or encapsulated in liposomes, as in immunoliposomes directed to specific cells. These lipid formulations can be administered topically, systemically, or delivered via aerosol. See, e.g., U.S. Pat. Nos. 6,149,937; 6,146,659; 6,143,716; 6,133,243; 6,110,490; 6,083,530; 6,063,400; 6,013,278; 5,958,378; 5,552,157.

### KITS

The invention provides kits comprising the compositions, e.g., the pharmaceutical compositions, nucleic acids, cells, of the invention. The kits also can contain instructional material teaching methodologies, e.g., how and when to administer the pharmaceutical compositions, how to apply the compositions and methods of the invention to imaging systems, e.g., computer assisted tomography (CAT), magnetic resonance spectroscopy (MRS), magnetic resonance imaging (MRI), positron emission tomography (PET), single-photon emission computed tomography (SPECT) or bioluminescence imaging (BLI). Kits containing pharmaceutical preparations (e.g., chimeric polypeptides, vectors, nucleic acids) can include directions as to indications, dosages, routes and methods of administration, and the like.

### EXAMPLES

The following example is offered to illustrate, but not to limit the claimed invention.

### Example 1

### In Vivo Targeting to Angiogenic Vasculature

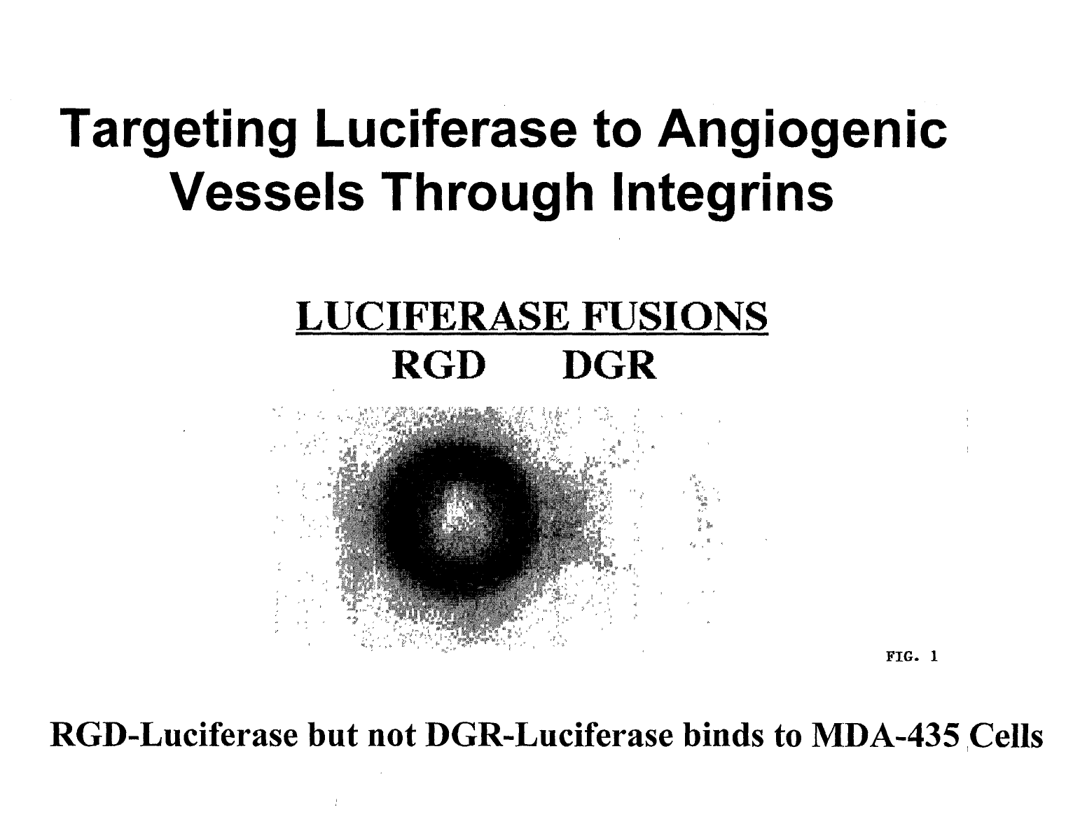

The following example demonstrates that the compositions and methods of the invention can be used as a molecular imaging approach to non-invasively detect neovascularization within tumors. An exemplary chimeric recombinant polypeptide of the invention, an RGD-containing-luciferase fusion protein was produced in a bacterial expression system. The RGD-luciferase protein and, as a negative control, the reverse sequenced DGR fusion protein (DGR-luciferase) were expressed. The RGD-luciferase protein had a strong tendency to multimerize (cyclize) due to reactive SH-groups on the terminal end of the peptide. This required production of the protein under dilute conditions. The recombinant proteins were purified and added to MDA-435 human breast carcinoma cells (see, e.g., Rahman (1989) J. Natl. Cancer Inst. 81:1794-1800) in culture wells. The MDA-435 cells, which express the RGD receptor on their cell surface, were useful for in vitro testing of this targeting approach for bioluminescent imaging (BLI). As shown in FIG. 1, the chimeric RGD-luciferase protein attached to the surface of the cells and could be detected using BLI. DGR-luciferase binding, serving as a control for possible non-specific binding, could not be detected.

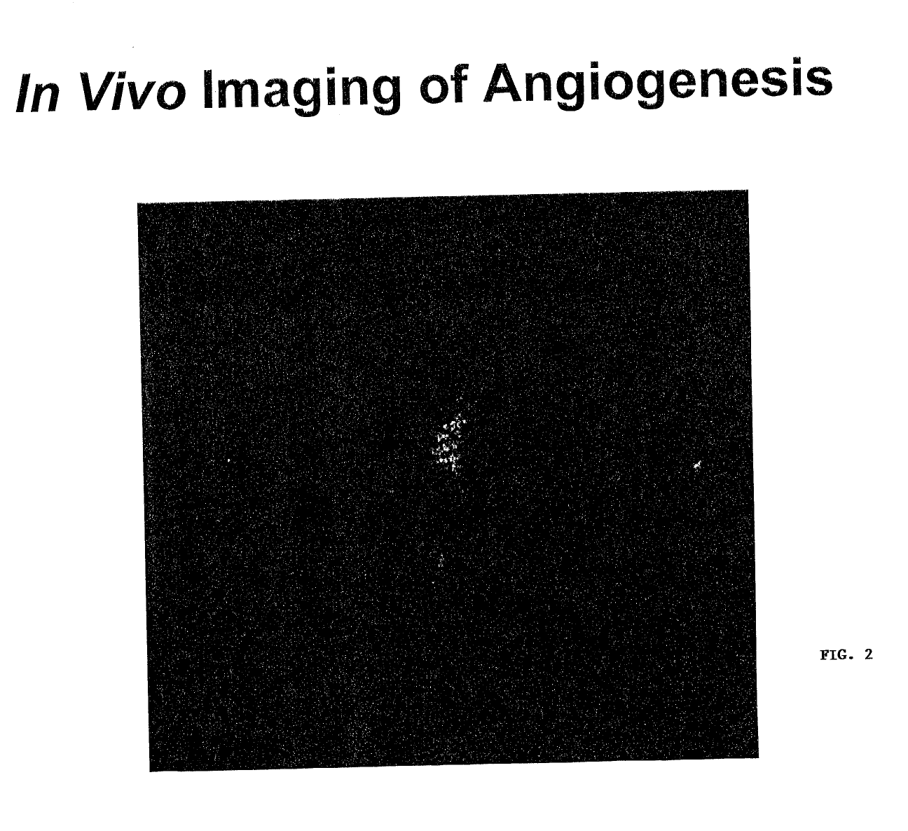

The RDG-luciferase was also injected into a nude mouse with an orthotopic mammary tumor. Luciferin was administered. The animal was imaged in an in vivo bioluminescent imaging system. As shown in FIG. 2, the presence of the tumor was detected by the emission of luciferase-produced photons from the tumor site (see arrow).

A number of embodiments of the invention have been described. Neverthe-less, it will be understood that various modifications may be made without departing from the spirit and scope of the invention. Accordingly, other embodiments are within the scope of the following claims.

What is claimed is:

1. A chimeric molecule comprising a first domain comprising a fluorescent, bioluminescent or chemiluminescent polypeptide, or a heterologous kinase, and a second domain comprising a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide.

2. The chimeric molecule of claim 1, wherein the chemiluminescent polypeptide comprises luciferase.

3. The chimeric molecule of claim 1, wherein the fluorescent, bioluminescent or chemiluminescent compound comprises an aequorin, an obelin, a mnemiopsin or a berovin.

4. The chimeric molecule of claim 1, wherein the heterologous kinase comprises a herpes simplex virus-1 thymidine kinase (HSV-1 TK).

5. The chimeric molecule of claim 1, wherein the RGD motif-comprising polypeptide specifically binds to a cell-specific polypeptide, a tissue-specific polypeptide or an organ-specific polypeptide.

6. The chimeric molecule of claim 5, wherein the cell-specific polypeptide is a tumor-specific polypeptide.

7. The chimeric molecule of claim 5, wherein the cell-specific polypeptide is expressed on tumor neovasculature.

8. The chimeric molecule of claim 1, wherein the RGD motif-comprising polypeptide comprises an integrin polypeptide.

9. The chimeric molecule of claim 1, wherein the RGD motif-comprising polypeptide comprises an CDCRGDCFC (SEQ ID NO: 1) amino acid sequence.

10. The chimeric molecule of claim 1, wherein the selectin is an E-selectin.

11. The chimeric molecule of claim 1, wherein the selectin binding polypeptide comprises an IELLQAR (SEQ ID NO:2) amino acid sequence.

12. The chimeric molecule of claim 1, wherein the matrix metalloproteinase is a gelatinase A or a gelatinase B.

13. The chimeric molecule of claim 1, wherein the matrix metalloproteinase binding polypeptide comprises an CTTHWGFTLC (SEQ ID NO:3) amino acid sequence.

14. The chimeric molecule of claim 1, wherein the chondroitin sulfate proteoglycan comprises a high molecular weight human melanoma-associated antigen.

15. The chimeric molecule of claim 1, wherein the chondroitin sulfate proteoglycan binding polypeptide comprises a TAASGVRSMH (SEQ ID NO:4) or LTLRWVGLMS (SEQ ID NO:5) sequence.

16. The chimeric molecule of claim 1, wherein the chimeric molecule is capable of specifically binding to a lumen-expressed vascular endothelial cell protein.

17. A gold nanoparticle comprising the chimeric molecule of claim 1.

18. A pharmaceutical formulation comprising a chimeric molecule and a pharmaceutically acceptable excipient,
wherein the chimeric molecule comprises a first domain comprising a fluorescent, bioluminescent or chemiluminescent polypeptide, or a heterologous kinase, and a second domain comprising a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide,
wherein the formulation is suitable for administration as an imaging enhancing agent and the chimeric molecule is present is an amount sufficient to enhance a computer assisted tomography (CAT) image, a magnetic resonance spectroscopy (MRS) image, a magnetic resonance imaging (MRI) image, a positron emission tomography (PET) image, a single-photon emission computed tomography (SPECT) image or a bioluminescence image (BLI).

19. A pharmaceutical formulation comprising
a composition comprising a first domain comprising an imaging enhancing agent and a second domain comprising a polypeptide that binds to a cell, a tissue or an organ in a cell-, tissue-, or organ-specific manner, and a pharmaceutically acceptable excipient,
wherein the formulation is suitable for administration as an imaging enhancing agent and the composition is not an antibody and is present is an amount sufficient to enhance a computer assisted tomography (CAT) image, a magnetic resonance spectroscopy (MRS) image, a magnetic resonance imaging (MRI) image, a positron emission tomography (PET) image, a single-photon emission computed tomography (SPECT) image, a bioluminescence image (BLI) or equivalent, when the pharmaceutical formulation is administered to an individual.

20. The pharmaceutical formulation of claim 19, herein the imaging enhancing agent comprises a fluorescent, a bioluminescent or a chemiluminescent polypeptide and the composition is a chimeric recombinant protein.

21. The pharmaceutical formulation of claim 20, wherein the bioluminescent or chemiluminescent compound comprises a luciferase.

22. The pharmaceutical formulation of claim 20, wherein the bioluminescent or chemiluminescent compound comprises an aequorin, an obelin, a mnemiopsin or a berovin.

23. The pharmaceutical formulation of claim 19, wherein the bioluminescent or chemiluminescent compound comprises a phenanthridinium ester.

24. The pharmaceutical formulation of claim 19, wherein the image enhancing agent comprises a kinase.

25. The pharmaceutical formulation of claim 24, wherein the kinase comprises a herpes simplex virus-1 thymidine kinase (HSV-1 TK).

26. The pharmaceutical formulation of claim 19, wherein the imaging-enhancing agent comprises a radioactive isotope.

27. The pharmaceutical formulation of claim 26, wherein the radioactive isotope comprises ¹³¹I, ¹²⁵I, ¹²³I, ¹⁸F, ¹¹C, ⁷⁵Br, ⁷⁶Br, ¹⁹F, ¹³C, ¹⁴C or ³H.

28. The pharmaceutical formulation of claim 19, wherein the imaging-enhancing agent comprises a paramagnetic compound.

29. The pharmaceutical formulation of claim 28, wherein the paramagnetic compound comprises a polypeptide chelated to a metal.

30. The pharmaceutical formulation of claim 28, wherein the paramagnetic compound comprises a metalloporphyrin.

31. The pharmaceutical formulation of claim 28, wherein the paramagnetic compound comprises a monocrystalline nanoparticle.

32. The pharmaceutical formulation of claim 31, wherein the monocrystalline nanoparticle comprises an iron oxide or a lanthanide.

33. The pharmaceutical formulation of claim 28, wherein the paramagnetic compound comprises a metal ion comprising a lanthanide of atomic numbers 58-70, a transition metal of atomic numbers 21 to 29 , 42 or 44, a Gd(III), a Mn(II), or an element comprising an Fe element.

34. The pharmaceutical formulation of claim 28, wherein the paramagnetic compound is a neodymium iron oxide (NdFeO₃) or a dysprosium iron oxide (DyFeO₃).

35. The pharmaceutical formulation of claim 19, wherein the imaging-enhancing agent comprises a synthetic compound.

36. The pharmaceutical formulation of claim 19, wherein the composition comprises a peptidomimetic or a peptide.

37. The pharmaceutical formulation of claim 19, wherein the compound of the second domain comprises a member selected from the group consisting of an RGD motif-comprising polypeptide; a selectin-binding polypeptide; a matrix metalloproteinase (MMP)-binding polypeptide, and a chondroitin sulfate proteoglycan-binding polypeptide.

38. The pharmaceutical formulation of claim 19, further comprising a cytotoxic agent.

39. The pharmaceutical composition of claim 38, wherein the cytotoxic agent comprises an antitumor agent.

40. The pharmaceutical composition of claim 19, further comprising a substrate for the bioluminescent or chemiluminescent polypeptide or the heterologous kinase.

41. The pharmaceutical composition of claim 40, wherein the chemiluminescent polypeptide is luciferase and the substrate is luciferin.

42. The pharmaceutical composition of claim 40, wherein the heterologous kinase is 8-[18F] fluoroganciclovir (FGCV) and the substrate is a herpes simplex virus-1 thymidine kinase (HSV-1 TK).

43. The pharmaceutical composition of claim 19, further comprising a liposome.

44. The pharmaceutical formulation of claim 19, wherein the pharmaceutically acceptable excipient is a buffered saline, or equivalent.

45. A nucleic acid encoding a chimeric polypeptide as set forth in claim 1.

46. A nucleic acid comprising an open reading frame operably linked to a promoter, wherein the open reading frame encodes a chimeric polypeptide as set forth in claim 1.

47. An expression vector comprising a nucleic acid encoding a chimeric polypeptide as set forth in claim 1.

48. A cell comprising a nucleic acid encoding a chimeric polypeptide as set forth in claim 1.

49. The cell of claim 48, wherein the cell is a bacterial, a yeast, an insect, or a mammalian cell.

50. A recombinant chimeric polypeptide produced by a cell comprising a nucleic acid encoding a chimeric polypeptide as set forth in claim 1.

51. A method for in situ or in vivo imaging of a cell, a tissue, an organ or a full body comprising administration of a pharmaceutical formulation in an amount sufficient to enhance the image, wherein the pharmaceutical formulation comprises a composition as set forth in claim 18,
wherein the image is generated by computer assisted tomography (CAT), magnetic resonance spectroscopy (MRS), magnetic resonance imaging (MRI), positron emission tomography (PET), single-photon emission computed tomography (SPECT), bioluminescence imaging (BLI) or equivalent.

52. A method for in situ or in vivo imaging of a cell, a tissue, an organ or a full body comprising the following steps:
(a) providing a pharmaceutical formulation as set forth in claim 18;
(b) providing an imaging device
wherein the imaging device is a computer assisted tomography (CAT) device, a magnetic resonance spectroscopy (MRS) device, a magnetic resonance imaging (MRI) device, a positron emission tomography (PET) device, a single-photon emission computed tomography (SPECT) device, a bioluminescence imaging (BLI) device or equivalent;
(c) administering the pharmaceutical formulation in an amount sufficient to generate the cell, tissue or body image; and,
(d) imaging the distribution of the pharmaceutical formulation of step (a) with the imaging device, thereby imaging the cell, tissue or body.

53. The method of claim 52, wherein the pharmaceutical formulation is administered to a human.

54. The method of claim 52, wherein the tissue is a tumor tissue.

55. The method of claim 52, wherein the pharmaceutical formulation is administered intravenously.

56. The method of claim 52, wherein image is taken between about 2 minutes and about 24 hours after administering the pharmaceutical formulation.

57. The method of claim 52, further comprising providing a substrate for the bioluminescent or chemiluminescent polypeptide, or the heterologous kinase, and administering the substrate with or after administration of the bioluminescent or chemiluminescent polypeptide or the heterologous kinase.

58. The method of claim 57, wherein the chemiluminescent polypeptide is luciferase and the substrate is luciferin.

59. A method for in vivo imaging a tumor neovasculature in an individual comprising the following steps:
(a) providing a pharmaceutical formulation as set forth in claim 18;
(b) providing an imaging device
wherein the imaging device is computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent;
(c) administering the pharmaceutical formulation in an amount sufficient to image the tumor neovasculature; and,
(d) imaging the distribution of the pharmaceutical formulation of step (a) with the imaging device, thereby imaging the tumor neovasculature.

60. A method for in vivo screening for an anti-tumor agent by imaging a tumor neovasculature in an individual comprising the following steps:
(a) providing a composition comprising a chimeric polypeptide as set forth in claim 1 or a pharmaceutical formulation as set forth in claim 18, and a test compound;
(b) providing an imaging device
wherein the imaging device is computer assisted tomography (CAT), magnetic resonance spectroscopy (MRS), magnetic resonance imaging (MRI), positron emission tomography (PET), single-photon emission computed tomography (SPECT), bioluminescence image (BLI) or equivalent;
(c) administering the composition of step (a) in an amount sufficient to image the tumor neovasculature and imaging the distribution of the composition with the imaging device, thereby imaging the tumor neovasculature;
(d) administering the test compound; and
(e) imaging the distribution of the composition with the imaging device, thereby imaging the tumor neovasculature, wherein a decrease in the amount of tumor neovasculature indicates that the compound is an anti-tumor or an anti-angiogenic agent.
